(19) United States
(12) Patent Application Publication
(10) Pub. No.: US 2005/0278027 A1
(43) Pub. Date: Dec. 15, 2005

(54) Annulus fibrosus stent

(76) Inventor: Edward Robert Hyde JR., Turlock, CA (US)

Correspondence Address: Edward R. Hyde, Jr., P.O. Box 3907, Turlock, CA, 95381, US

(21) Appl. No.: 11/150,798

(22) Filed: Jun. 10, 2005

Related U.S. Application Data
(60) Provisional application No. 60/521,653, filed on Jun. 11, 2004.

(51) Int. Cl. A61F 2/44

(52) U.S. Cl. 623/17.12; 623/17.11; 623/17.16

(57) ABSTRACT

The Annulus Fibrosus Stent (AFS) is a platform of barriers used in the intervertebral disc. The barrier can be inserted into an intervertebral disc to act to reinforce and/or supplement the disc. The AFS can be inserted at any of the many different stages of disc pathology. The AFS can be inserted to prevent pressure and dissection of disc material which in and of itself can decrease and/or eliminate pain from damaged AF fibers. The AFS can be inserted before disc pathology progresses to a substantial event such as a disc herniation. It can be inserted at any time in the progression of the natural history of disc disease. It can even be inserted when substantially the entire intervertebral disc has been removed. It can be inserted in conjunction with another procedure such as a Nucleus Pulposus Replacement (NPR) or Total Disc Replacement (TDR), etc. The various shapes and material of the AFS in this patent are designed to address particular clinical situations, particular anatomy and particular stages of disc degeneration. The AFS can be inserted directly through the AF, or by a technique of detaching part of the AF with bone without actually cutting a substantial part of AF fibers or layers of fibers or by the Transosseous approach.

### BACKGROUND OF INVENTION

This application claims benefit of provisional application ANNULUS FIBROSUS STENT Ser. No. 60/521653 that was filed on Jun. 11, 2004 19:14 EDT.

Pathology of the spine accounts for substantial medical costs, disability and reduced quality of life. Serious disease affects patients at a much younger age than other joint pathology such as knee and hip arthritis. Herniated nucleus pulposa (HNP) accounts for a large proportion of spine pathology. The HNP has several consequences. Initially the HNP causes pain due to torn fibers of the annulus fibrosus (AF) and pressure on soft tissues and nerves. This can lead to neurological sequlae such as loss of sensation, pain, loss of DTR and motor weakness. Severe cases can cause disruption of bowel and bladder function, which require immediate surgical intervention. Secondarily the loss of the NP causes the intervertebral disc height to decrease leading to mal-alignment of the facet joints. This can lead to pain and eventual arthritis of the facet joints and osteophyte formation. The loss of the NP also changes the biomechanics of the disc leading to degenerative changes of the annulus fibrosus. Surgical treatments have been disectomy and/or fusion of adjacent spinal elements. More recently there has been an interest in disc replacements (TDR), facet replacements and replacement of the NP. None of these products are approved for general use in the United States at this time. Some TDR have had wide use in Europe. NP replacements have been made of many different types of materials and have various fixation methods. Hydrogels are a current concept under investigation. Polyurethane is also being explored. NP replacements have shown a tendency to herniate through the scar of the old annulus tear or through the implantation incision in the AF.TDR also compromise the AF and an AFS can be utilized in association with the TDR to limit migration of components. A mechanical Annulus Fibrosus Stent AFS is the focus and scope of this patent. The AFS is inserted and/or deployed inside the disc at a relative border between the NP and the AF. The AFS's role is to substantially reinforce or substitute for the AF across the majority of the disc, preventing herniation of material from other sites that might be weakened. It can also be used to provide scaffolding for AF and NP replacements, especially bioengineered organic materials. The AFS stent is a device that covers a substantially larger area than a patch for a defect in the AF that is limited to obliterating the defect. In some preferred embodiments it is completely circumferential along the inside diameter of the annulus fibrosus.

### SUMMARY OF INVENTION

The Annulus Fibrosus Stent (AFS) is designed to reinforce the annulus fibrosus (AF) from the inside of the AF to prevent herniation of NP, TDR or NPR that have been implanted in the disc space.

A type of patch has been proposed by Cauthen in multiple patents and patent applications. Suddaby suggests a vertical stent. Thomas suggests a collection space occupying implants. Other stent patch techniques have been proposed. These inventions address a tear or defect in the AF and a method of occluding the tear site without addressing the integrity of the rest of the disc or large defects that are made by surgery.

The AFS is designed to reinforce the AF along and/or throughout it internal limit or approximate boundary with the NP but more importantly to also prevent herniation of the NP, TDR or NPR or other material.

After a small disc herniation or even pre-herniation of a damaged disc an AFS can be inserted to prevent or prevent herniation or limit further herniation of material (native disc material). More severely compromised discs can use AFS to augment the disc alone or in conjunction with other procedures such as NP replacement or TDR.

The AFS can have several forms that are designed to address specific clinical needs and problems and be implanted by several methods.

The ASF implants will be divided into groups by method of implantation. They will be divided into those inserted by the transannulus approach and transosseous approach.

The transannulus method implants the AFS through the AF. It can be implanted through an incision in the AF, through the herniation site or the disc attachments can be dissected off the vertebral body with or without a bone piece or remnant and then reattached.

The transosseous method implants would utilize an implantation method that goes through the bone of a vertebral body to the footprint of the NP without disrupting the AF or its attachments. (See Hyde—TOSCA—Patent Pending)The transannulus implants can be expandable, deployable or inserted in units or modules and assembled in vitro. A NP or TDR can be implanted in conjunction with a AFS.

The AFS can be designed in any number of forms as long as it accomplishes the task of becoming a barrier to internally reinforce the AF or remaining portion of the AF. They can be made of any biocompatible material or combinations of biocompatible material. The AFS can be modular.

Some of the types of AFSs are listed below and included in the figures.
1)Coil
2)Sheet
3)Rods/Bars/Pins
4)Balloon/Bladder
5)Plates Curved, Straight
6)Washers
7)Mesh/Net
8)Cage/Basket
9)Basket with or without closure
10)Collapsible frame with lockable hinged connectors
11)Interlocking Sheets
12)Pleated Sheet
13)Rib/Slat
14)Spray
15)Putty
16)Ductile Metal
17)Low Temperature Metal
18)Porcelain composite
20)Liquid that cures

### BRIEF DESCRIPTION OF DRAWINGS

FIG. 1A: Coil Annulus Fibrosus Stent

FIG. 1B: Coil Annulus Fibrosus Stent Pitch Variation

FIG. 2A: Sheet Annulus Fibrosus Stent Rolled

FIG. 2B: Sheet Annulus Fibrosus Stent Rolled and Deployed

FIG. 3: Inflatable Annulus Fibrosus Stent Holes & Spikes

FIG. 4: Inflatable Annulus Fibrosus Stent Rolled Spiral

FIG. 5: Modular Sheet Annulus Fibrosus Stent

FIG. 6: Cage/Rebar Annulus Fibrosus Stent

FIG. 7: Disc Annulus Fibrosus Stent Aligned

FIG. 8: Disc Annulus Fibrosus Stent Staggered

FIG. 9: Basket Annulus Fibrosus Stent with Aperture

FIG. 10: Perforated Basket Annulus Fibrosus Stent with Open Ends

FIG. 11: Perforated Basket Annulus Fibrosus Stent with Aperture & Lid

FIG. 12: Interlocking Sheets Annulus Fibrosus Stent Long Axis

FIG. 13: Interlocking Sheets Annulus Fibrosus Stent Short Axis

FIG. 14: Interlocking Sheets Annulus Fibrosus Stent Long & Short Combined

FIG. 15: Pleated Sheet Annulus Fibrosus Stent

FIG. 16: Pleated Sheet Annulus Fibrosus Stent with Locking Rings

FIG. 17: Rib/Slat Annulus Fibrosus Stent

FIG. 18: Rib/Slat Annulus Fibrosus Stent with Rings—Collapsed

FIG. 19: Rib/Slat Annulus Fibrosus Stent with Rings Deployed (18)

FIG. 20: Rib/Slat Annulus Fibrosus Stent with Rings Deployed (36)

### DETAILED DESCRIPTION

GENERAL PROPERTIES The following statements and properties apply to all AFS independent of their shape, design and method of installation. Many of the AFS are able to be inserted through minimally invasive approaches.

Annulus Fibrosus Stent (AFS)The AFS can be inserted as a preventative device before complete disc herniation. It will prevent herniation and also decrease pressure at the weakened portion of the AF reducing or eliminating pain from dissecting NP or other concentrated pressures on a disc defect. Minimally invasive NP replacements can be used in conjunction with AFSs to reconstitute disc function.

Cases that have substantial disc pathology or herniation of material additional material might need to be removed. Any amounts of the NP and AF can be removed before implantation of the ASF. The inserted AFS will provide a boundary or partial boundary. The preferred embodiment will be an AFS that will be substantially round or elliptical. The AFS can be any geometric shape and having an inner and outer surface with a void or cavity forming an enclosure or partial enclosure. The inner and or outer surfaces forming parietal structures. The AFS can be symmetric or non-symmetric in any plane. The size and shape of the AFS will be chosen based on the surgeon's choice of placement, the pathology and the anatomy.

Situations where substantially a major portion of the NP is removed or has been destroyed, a replacement NP might also be inserted in conjunction with the AFS. The AFS decreases the likelihood that the new NP replacement would herniate or dislodge.

This is especially true if a portion of the AF is removed as well. The AFS can be implanted before, after or simultaneously with the replacement NP.

The AFS can be used with artificial AF replacements or supplements, etc., organic or inorganic. It can act as a temporary or permanent superstructure or scaffolding for bioengineered AF or NP.

Situations where a disc replacement is implanted will also benefit from placement of an AFS. The TDR will be constrained from dislodging or migrating especially when part of the AF is also removed.

The AFS can be made of any substantially flexible material. It can be made of a material that can be easily shaped into a compressed from or from an elongated form that resumes a shape after implantation.

The AFS can be made of any bio-compatible material: metal, plastic, ceramic, organic, carbon-based material, etc. It can be made of polyurethane. The AFS can be modular. The AFS can be collapsible and deployable. The AFS can be made of laminates. Laminates can be solids and/or liquids.

The AFS can be honeycombed or have an open patterned matrix. The AFS can have interlocking ribs or slats. The interlocking rib ASF can be collapsible and deployable.

Shape Memory Alloys (SMA) and Shape Memory Plastics (SMP) are favorable materials for preferred embodiments. Any other Shape Memory materials can be used.

The AFS can be a geometric shape that has its pattern cut out of a sheet of SMA or SMP. The SMA/SMP is then folded to shape using at one temperature above the Austenite Finish temperature and then unfolded at another temperature. The SMA/SMP then returns to the folded shape when warmed to the previous temperature. A SMA such as Nitinol proceeds from the Austenite to the Martensite and back to the Austenite state and shape.

The AFS can be made of a material that can be reabsorbed or partially reabsorbed over time based on its constituent materials. The AFS can be a composite of more than one material such as a SMA and a material that can be reabsorbed (i.e. glycolic acid, lactic acid etc.). The AFS can be coated with materials (i.e. Hydroxyapatite, etc. or surface textures (i.e. beads, fibers, etc.) to promote functions such as fixation or ease of insertion. Pharmaceuticals can be attached to the AFS. The AFS can be manipulated by extra-corporal energy sources. The AFS can have a power source or a capacity to receive and store energy. Energy can be used to change the shape or enhance the functionality of the AFS after implantation.

The AFS can absorb and or dissipate energy or the AFS can be static. The AFS can damp transmission of energy.

The AFS can be incorporated into devices with other functions. The AFS can be modular and/or hybrids of materials. The AFS can include magnetic material or material that can be temporally magnetized. Magnetic material can be used to manipulate the AFS, absorb energy, provide fixation, etc.

The AFS can have fixation elements that function to restrain movement and or provide or enhance fixation to bone or soft tissue.

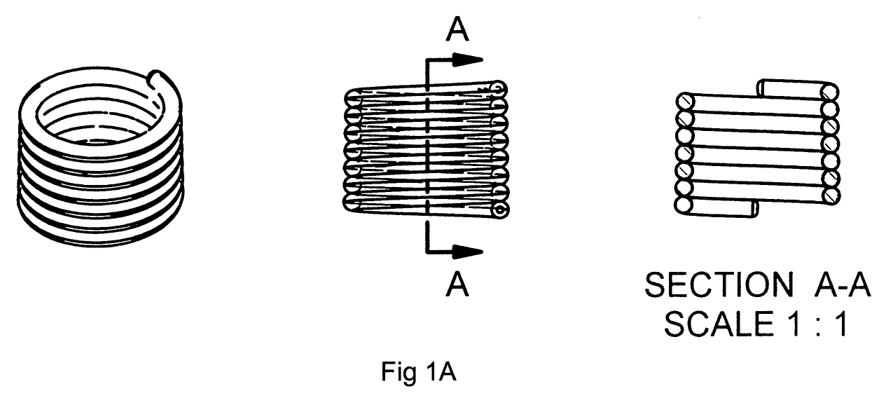

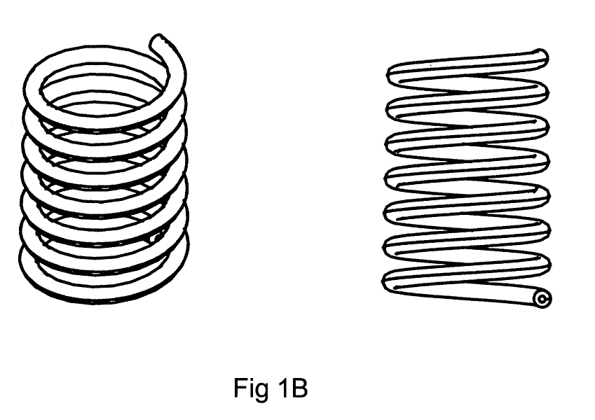

FIGS. 1A & 1B show a Coil type AFS. There are three views: isometric, normal and section. The relative shape and diameter of the Coil type AFS is sufficient to substantially place a barrier at a boundary between a natural or damaged nucleus pulposa (NP) and the natural annulus fibrosus (AF) of an intervertebral disc. The size and shape will be relative to the spine level and patient size and anatomy. The choice of the boundary position will depend on the surgeons assessment of the clinical situation and anatomy. A Coil type AFS can be inserted at or into this approximate boundary by inserting it in an uncoiled fashion through the AF as an elongated material. The elongated material would then form a coil as it is inserted or after it was inserted. The coil would penetrate the natural substances of the disc forming a plane substantially between or at the transition zone between NP and AF. Insertion would require a minimal opening in the AF to insert this AFS, just large enough to implant the uncoiled elongation. One preferred embodiment utilizes an instrument that contains a cannulated tube that acts to guide and shape the coil. The instrument is placed at the appropriate level in the disc and at the appropriate depth at the inner portion of the AF and the NP. The Coil type AFS can also be implanted into the disc through other methods such as the transosseous approach. (HYDE—U.S. Patent) The Coil type AFS can also be inserted en block as a pre fabricated coil with or without winding or unwinding. FIG. 1A shows a Coil type AFS (with each loop substantially touching the other loops above and below them. The Coil type AFS can be made such the loops are not touching. (FIG. 1B) The Coil type AFS can have more than one coil or helix. The coils or helices can have different handedness. (i.e. left/right) (Not shown). The coils or helices can be made of different materials or materials with different mechanical, physical, chemical, etc., properties. The coil can also be or contain a hollow elongation that can be inflated or filled with a gas or liquid (Not shown). The liquid can then cure or harden. The Coil type AFS can have additional terminal features or fixtures to increase functionality especially fixation such as hooks, barbs, protrusions, etc. Additional elements such as pegs, rods and screws can be used to provide or augment fixation (Not shown). Cement can be used if indicated. The Coil type AFS can be a superstructure for a fabric, mesh, screen, fibers, etc. that act as part of the barrier when the coil is deployed.

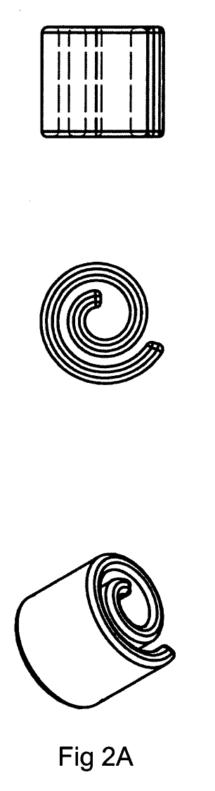

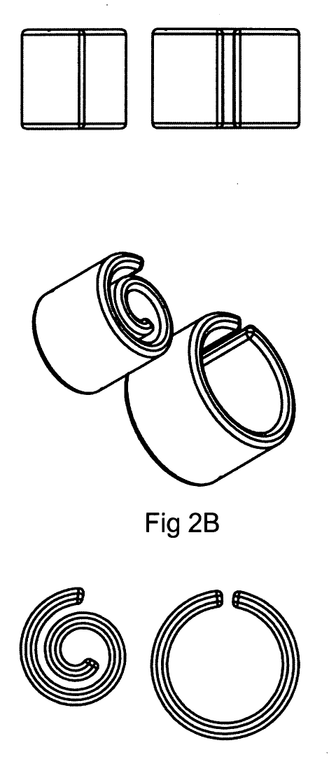

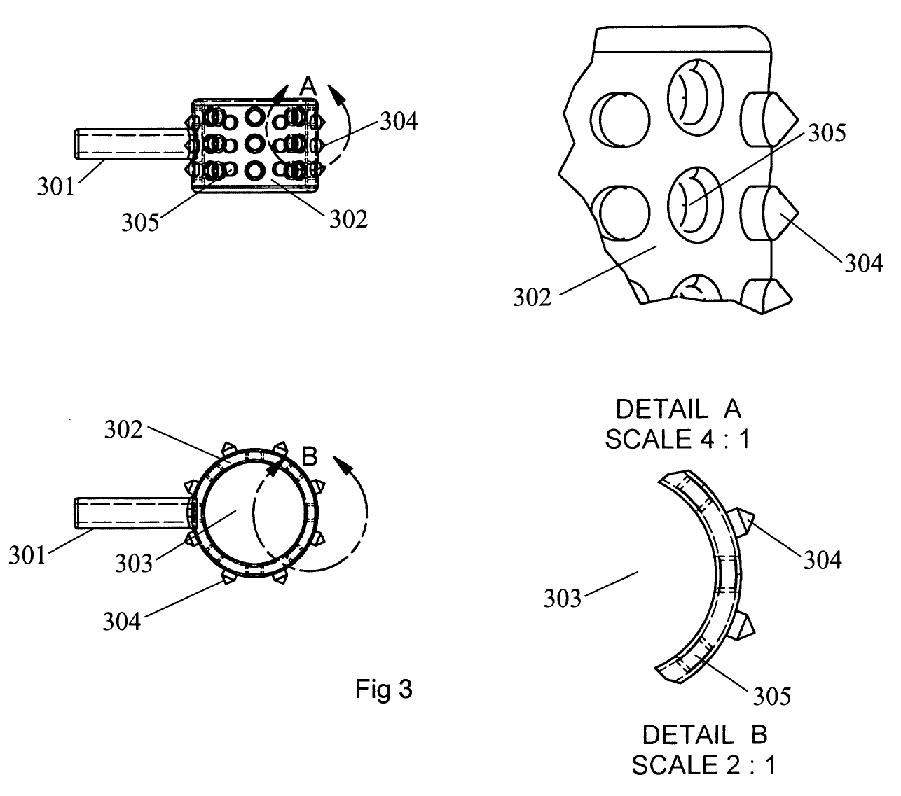

FIG. 2A shows an AFS in the shape of a sheet that is rolled up. The Sheet AFS is shown as a single piece. The AFS sheets can be modular or can be used to build a modular AFS. The modular units can be used to increase the height (i.e. strips that are assembled)(Not shown)or used to thicken the AFS by placing each layer front to back (Not shown). The Sheet AFS can be solid or they can be hollow (i.e. a bladder). The hollow Sheet AFS can be filled with a gas or liquid. The liquid can turn hard after filling the sheet (i.e. PMMA, polyurethane). The Sheet AFS can be perforated (Not shown See FIG. 3). The Sheet AFS can have protrusions or textures to increase functionality (Not shown See FIG. 3).The Sheet AFS can be substantially a complete boundary (i.e. with deployed ends substantially touching) or a partial boundary preferably greater than half the circumference or boundary.

FIG. 2B shows a Sheet type AFS rolled and deployed. balloon

FIG. 3 shows a Bladder type deployable and substantially hollow AFS. There is an elongation 301 to deploy the Bladder AFS with a liquid or gas. The Bladder AFS 302 is shown deployed. It can be collapsed into a smaller volume for insertion. The walls of the Bladder AFS can expand as well as deploy. The Bladder AFS walls in this particular embodiment are shown perforated 305 and have protrusions 304 to increase functionality. The gas or liquid inflated Bladder AFS can have a valve to retain the gas or liquid under pressure. Any biocompatible gas or liquid can be used. The Bladder AFS can be filled with a substance that hardens from its liquid form to make a AFS composite such as PMMA or any curable plastic, ceramic, polyurethane, etc. The Bladder AFS can be reinforced in or on the walls with thickenings or additional materials such as metals, plastics or carbon-based materials, etc.

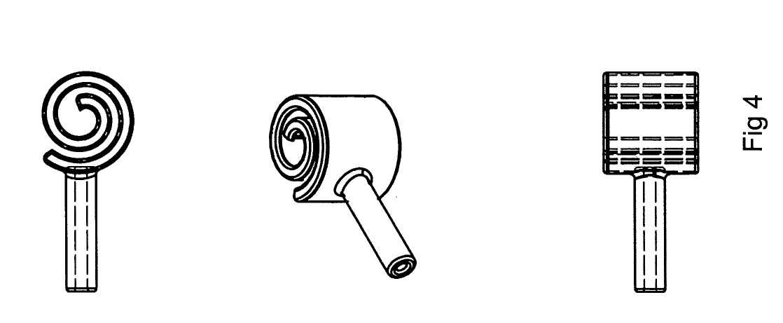

FIG. 4 shows another deployable Sheet AFS. It is a deployable substantially hollow AFS. It is shown not deployed. There is an elongation to deploy the Sheet AFS with a liquid or gas. The Sheet AFS can be deployed mechanically. The Sheet AFS walls can expand. The Sheet AFS walls can be perforated and have protrusions to increase functionality. The gas or liquid inflated Sheet AFS can have a valve to retain the gas or liquid under pressure. Any biocompatible gas or liquid can be used. The Sheet AFS can be filled with a substance that hardens from its liquid form to make a Sheet AFS composite such as PMMA or any curable plastic, ceramic, polyurethane, carbon based product etc. The Sheet AFS can also be reinforced with alternate materials.

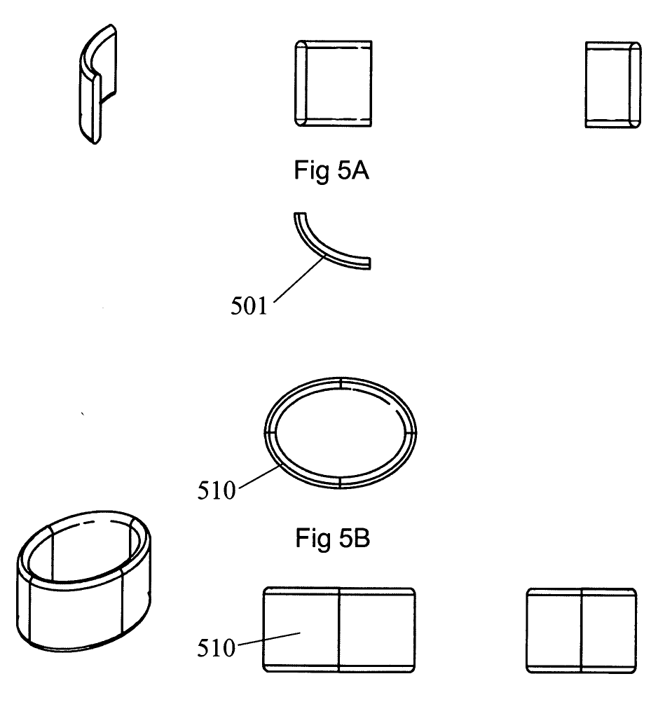

FIG. 5 shows a segmental Sheet AFS. It is composed of modular units 501 that are inserted in the disc space and then assembled. The figures shows an elliptical Sheet AFS 510 made of four equal sections. The segments are drawn as solids. Attachment mechanisms are not shown. Magnetic patterns in each unit can be used to auto assemble the units. The magnetic patterns are such that the units can assemble only in one fashion. Any other assembly method can be used. (i.e. Pegs and holes, tongue and groove, etc.)

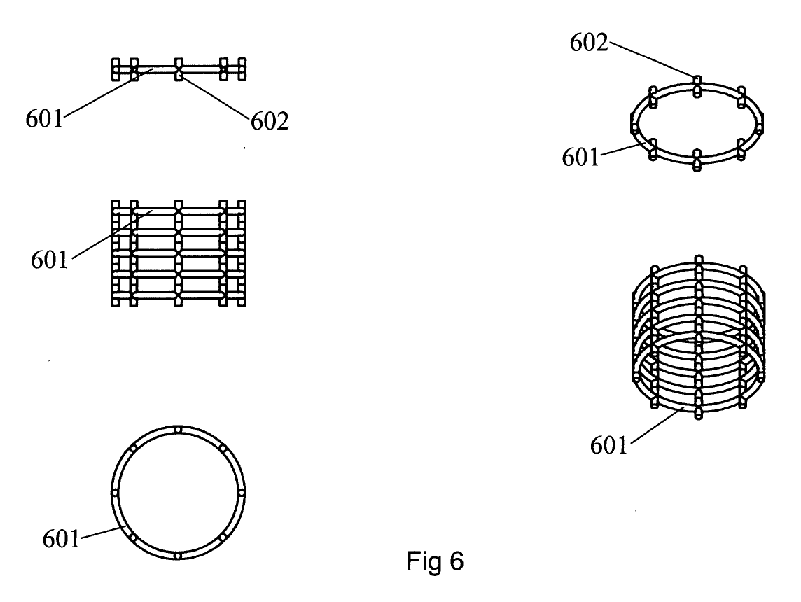

FIG. 6A and 6B show a Cage ASF. The Cage type AFS can be assembled from rings 601 or formed as a cage that can be compressed and then resume its deployed shape. Collapsible struts (Not shown) that lock when deployed can be used to allow a rigid structure after deployment. The cage can be made of a SMA, SMP or SMM to facilitate insertion and deployment. The modular rings can be connected by magnets or any other method known to the art.

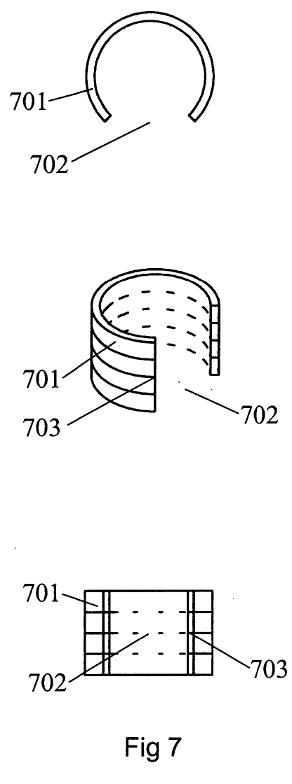

FIG. 7 show an AFS made of modular curved components 701 with the ability to align the elements in several modes. Here four stacked components are shown. Methods of attachment are not shown. The open sections 702 are all aligned. This allows insertion of objects or materials into the center of the enclosure formed by the curved components. The rings can be held together by magnetic forces or any other method known to the art.

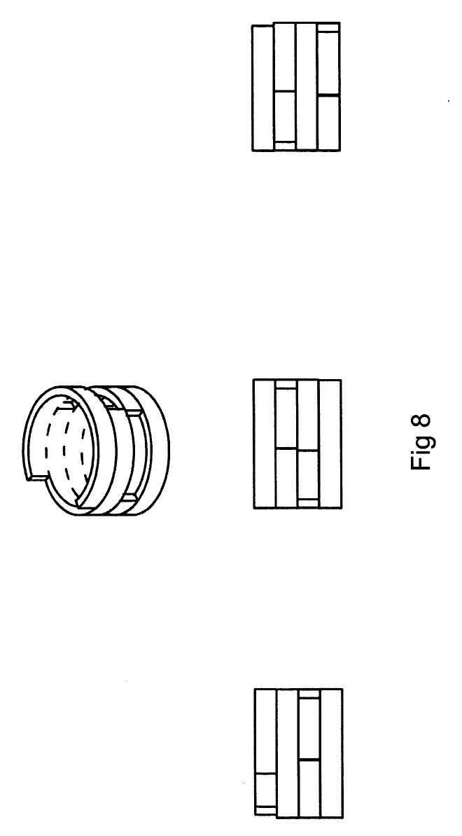

FIG. 8 shows the curved components form FIG. 7 orientated or staggered in a fashion to block the movement of anything in or out of the assembly. Rings can be locked once they are set in the staggered pattern.

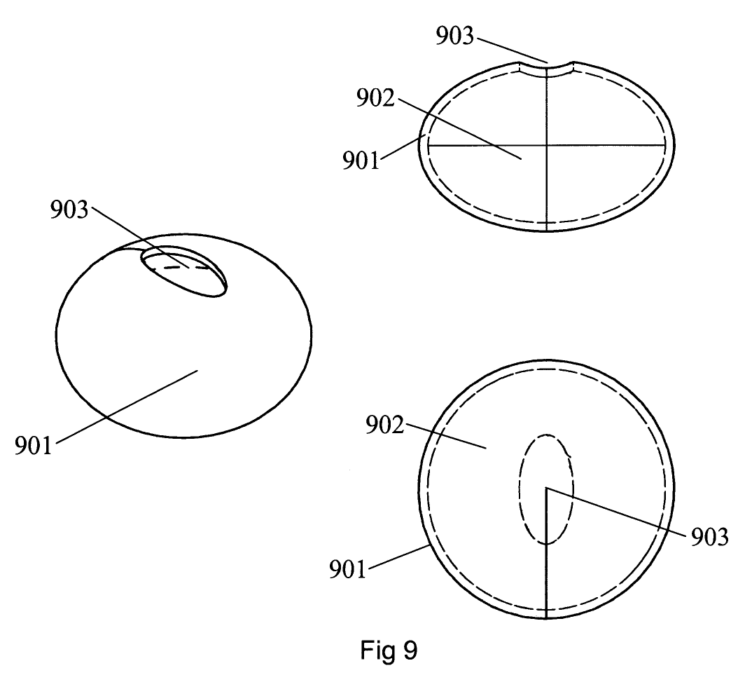

FIG. 9 shows a Basket type AFS 901. The Basket AFS is preferably flexible or collapsible to facilitate insertion into the disc. There is an aperture 903 to insert a NP, etc. The basket can be a mesh or fenestrated. The center 902 is hollow.

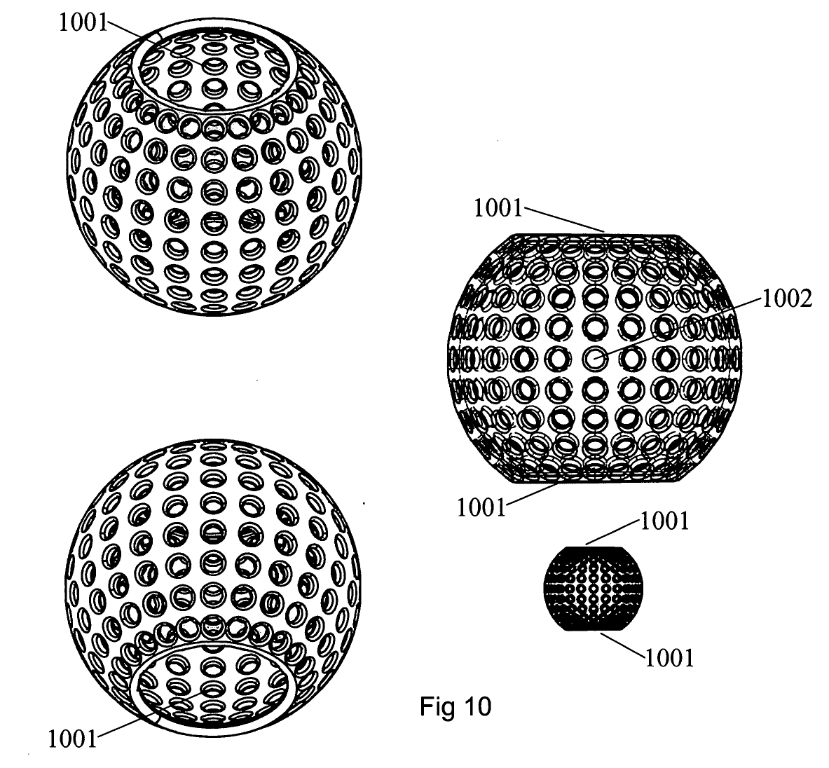

FIG. 10 shows a basket type AFS without a top or bottom 1001. It is fenestrated 1002. It can be an inflatable device. Large apertures can be used to insert NP, TDR, etc. It can be a rigid structure. It can be compressible. It can be elastic or viscoelastic. It can be anisometric in its direction or directions of compressibility.

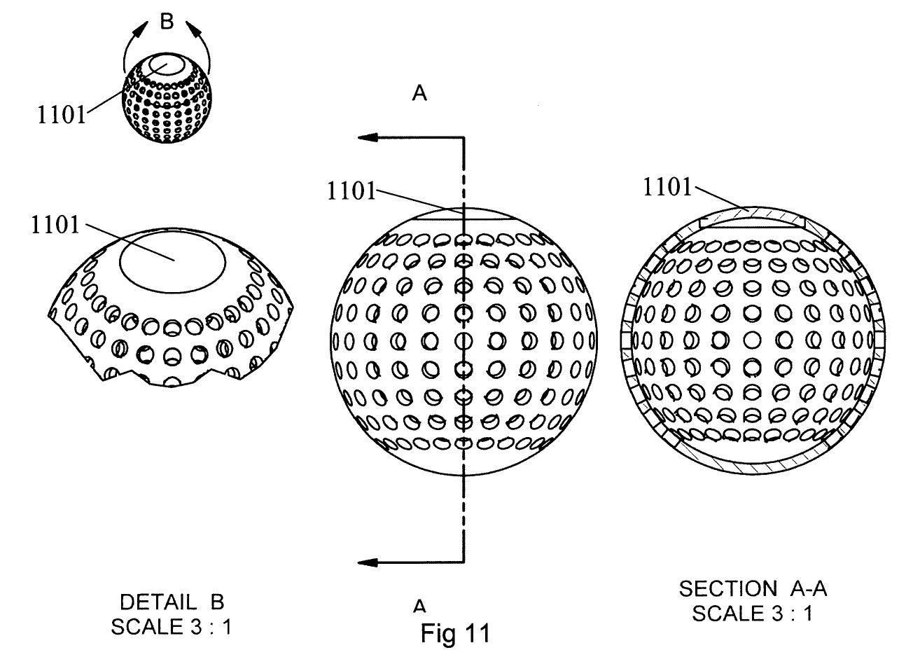

FIG. 11 shows a basket type AFS with a lid or closure device 1101. It can be rigid or resilient. It can be a mesh or fenestrated. It can be inflatable.

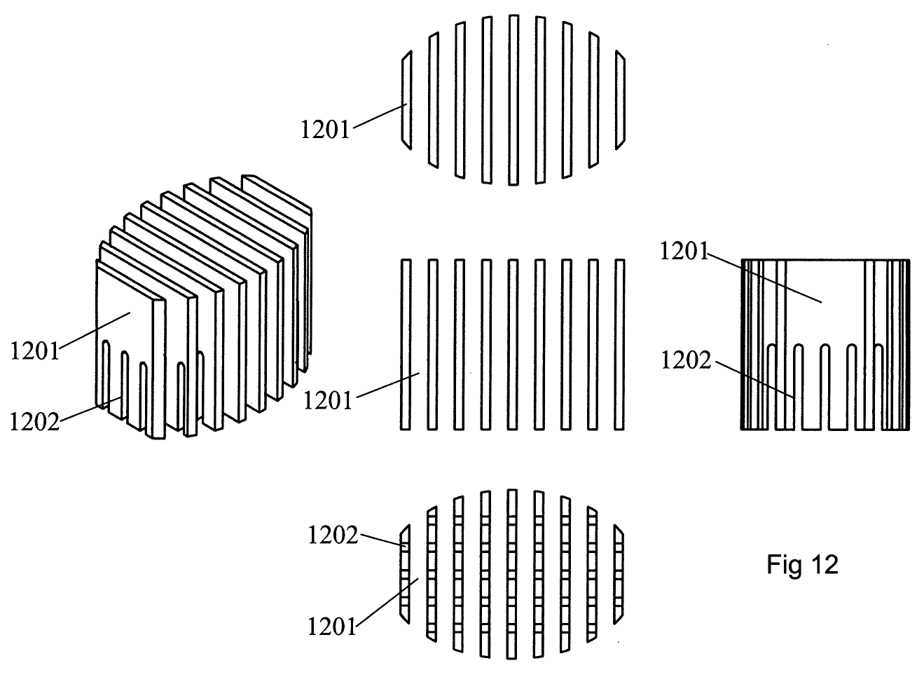

FIG. 12 shows ribs/slats (1201) perpendicular to the long axis of an elliptical device. Nine ribs/slats are shown. They are equally spaced. The ribs/slats can be any particle number. The pattern does not need to be uniform nor symmetric. There are slits 1202 cut out to enable these slats to interlock with other slats substantially perpendicular to these. Any pattern of slits or cut outs that allows of interlocking of the elements and the ability to collapse and then deploy can be used.

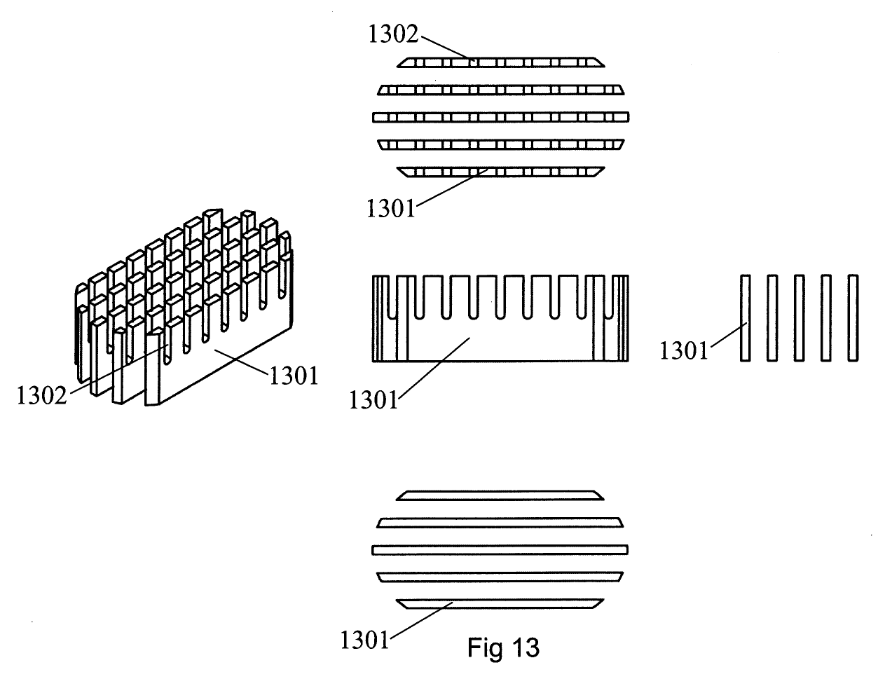

FIG. 13 shows five slats 1301 perpendicular to the short axis of the ellipse. These likewise have slits 1302 cut out to permit interlocking with the other slats shown in FIG. 12.

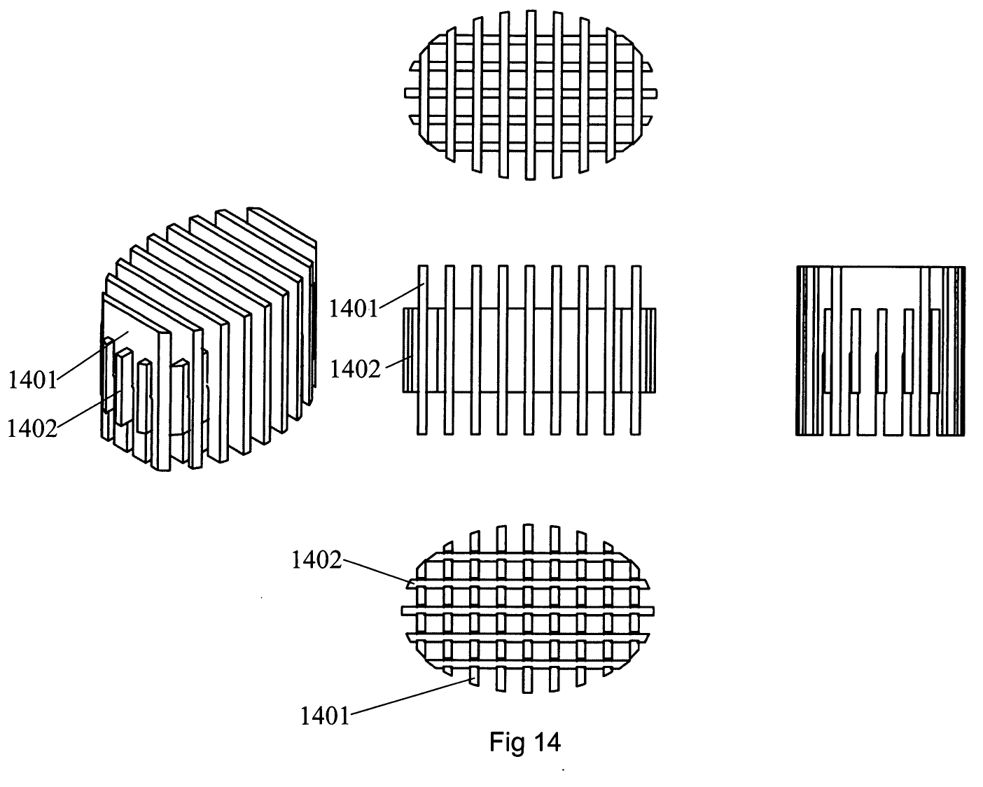

FIG. 14 shows the two sets assembled. They are shown deployed. They can be flattened or collapsed and then deployed. Once the interlocking slats are deployed they lock in place. The Locking mechanism is not shown. The slats can be reinforced or have flexible attachments to the adjacent slats to increase structural integrity or ease of assembly or deployment.

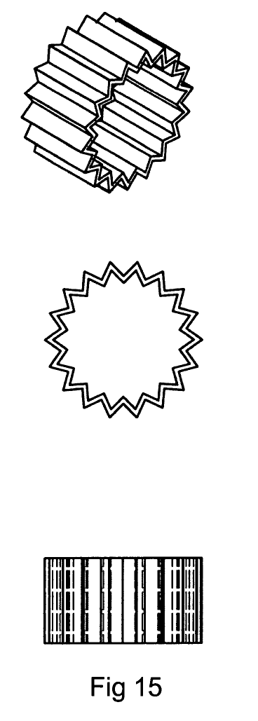

FIG. 15 shows a Folded AFS. The Folded AFS is deployed from a folded sheet into a circular arrangement. The Folded AFS can be deployed into any shape. The Folded AFS can be a continuous element or it can be a sheet that is deployed and then connected to itself. It can be deployed in a serpentine fashion with the ends not connected. It can have other structural elements or rods etc. to maintain the deployed shape (Not shown).

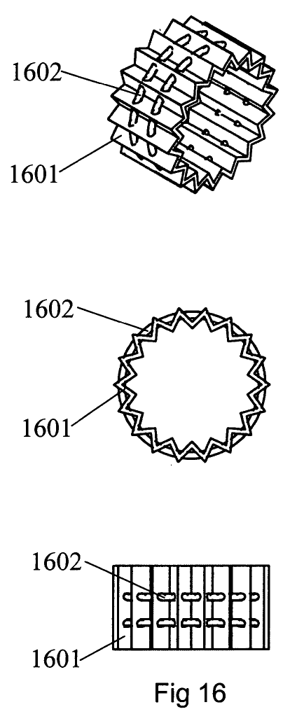

FIG. 16 shows a Folded AFS 1601 with wires or rings 1602 to maintain the deployed shape. Fastening mechanism of the Folded AFS to itself not shown. The wire or ring fastening mechanism and mechanism to attach the rings to the Folded AFS are not shown.

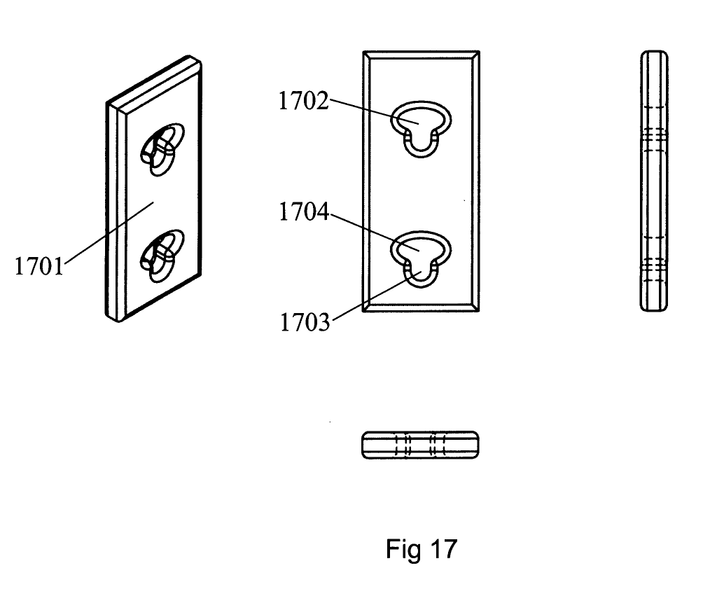

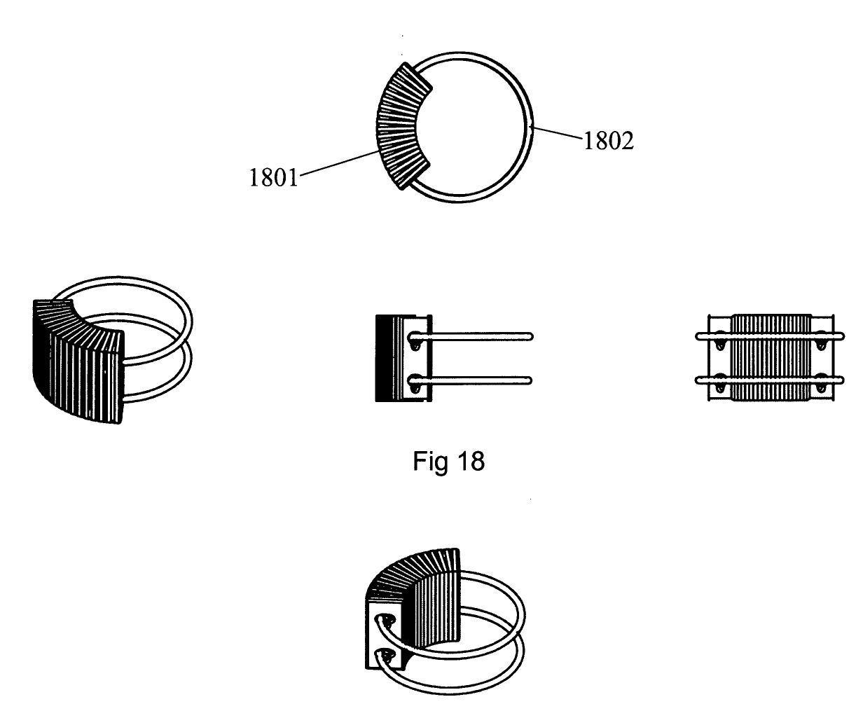

FIG. 17 shows a slat or rib (1701). There are two perforations in this embodiment 1702 and 1704. Each perforation in this particular embodiment has an elliptical top 1702, 1704 and a round bottom 1703. The Elliptical portion allows the slats to move and collapse. The round bottom portion locks the slats in a substantially radial orientation to the rings.( Shown in FIG. 18) The slats can also be locked in place.

FIG. 18 shows eighteen slats (1801) collapsed on two rings (1802). The wires or rings can be placed separately from the slats. They can be flexible to allow bending or deformation for insertion.

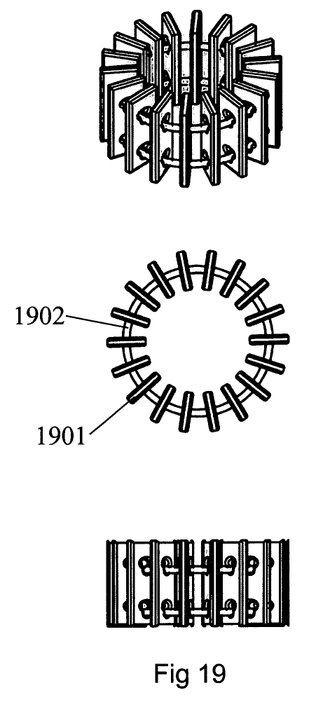

FIG. 19 shows the 18 slats (1901) deployed on two rings (1902).

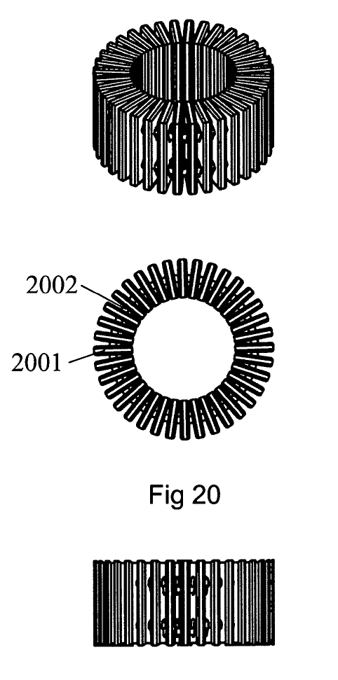

FIG. 20 shows 36 slats (2001) deployed on two rings (2002)

1) a device to be inserted into a intervertebral disc that substantially forms wall, barrier or reinforcement parallel to the vertical axis of the disc annulus fibrosus, internal to the outer boundary of the annulus fibrosus, that spans part of the disc height, preventing or inhibiting the movement of natural or synthetic material from an internal region of a disc to the outside of a disc.

2) the device of claim 1 that is substantially a thin solid tube of material, wire or line coiled in any chosen diameter smaller than the natural boundary of a disc in the axial plain

3) the device of claim 1 that is a sheet of material that can be rolled up and then deployed

4) the device of claim 1 where the device is deployable such as a bladder and can be filled with a gas, liquid or another material that cures to a solid

5) the device of claim 1 where the device can have projections or cavities on the surface to promote stabilization

6) the device of claim 1 where the device is modular and can be assembled by inserting individual parts into the disc and assembling the device

7) the device of claim 6 where the modular elements can be rings and the assembled device a cage made from individual elements

8) the device of claim 1 where it can be a substantially hollow ellipsoid or bladder that can be deployed after insertion

9) the device of claim 8 where there is a aperture to insert additional devices

10) the device of claim 8 where the hollow ellipsoid can have perforations

11) the device of claim 1 where the device can be made several interlocking pieces that can be assembled outside the disc and then deployed after insertion

12) the device of claim 1 where the device can be folded in an multifold pattern and the unfolded after insertion

13) the device of claim 12 where the unfolded device can be reinforced by additional structures such as rings or other suitable reinforcing structures

14) the device of claim 1 where it can be made of a shaped memory material

15) the device of claim 14 where it can be a shaped memory plastic

16) the device of claim 14 where it can be a shaped memory alloy

17) the device of claim 1 that can be coated with materials to promote fixation

18) the device of claim 1 where the device is not completely circumferential

19) the device of claim 1 where the device extends substantially from the vertebral body above the disc to the vertebral body below the disc

20) the device of claim 1 where it can be a mesh, net or cage that can be compressed, inserted and then deployed after insertion
